SEQUENCE LISTING (4 sequences)

SEQ ID NO: 1 - RNA, 19 nt, Artificial Sequence
  Description of Artificial Sequence Synthetic oligonucleotide
acaucaugauagccguaua

SEQ ID NO: 2 - RNA, 19 nt, Artificial Sequence
  Description of Artificial Sequence Synthetic oligonucleotide
aaaucuaagucaagcggaa

SEQ ID NO: 3 - RNA, 19 nt, Artificial Sequence
  Description of Artificial Sequence Synthetic oligonucleotide
acacaaccgaggagcguau

SEQ ID NO: 4 - RNA, 19 nt, Artificial Sequence
  Description of Artificial Sequence Synthetic oligonucleotide
cggagaagcccucgagcau

(19) United States
(12) Patent Application Publication
(10) Pub. No.: US 2014/0179688 A1
(43) Pub. Date: Jun. 26, 2014

(54) PLOD-2 Modulators and Their Use in the Treatment of Skin

(71) Applicant: AVON PRODUCTS, INC., Suffern, NY (US)

(72) Inventors: Uma Santhanam, Tenafly, NJ (US); Permanan Raaj Khusial, Highland Mills, NY (US)

(73) Assignee: AVON PRODUCTS, INC., Suffern, NY (US)

(21) Appl. No.: 13/721,687

(22) Filed: Dec. 20, 2012

(51) Int. Cl. A61K 8/49; A61Q 19/08; A61Q 19/00

(52) U.S. Cl. CPC A61K 8/49; A61K 8/4966; A61Q 19/00; A61Q 19/08
USPC 514/218; 514/242; 435/6.12

(57) ABSTRACT

Methods for preventing, ameliorating, or reducing dermatological signs of aging are provided which employ active agents that modulate the PLOD-2 enzyme in the skin. Also provided are methods for screening for substances which modulate PLOD-2 enzyme levels and the methods of using active agents identified by the screening protocol in the treatment of skin.

### SEQUENCE LISTING

The instant application contains a Sequence Listing which has been submitted in ASCII format via EFS-Web and is hereby incorporated by reference in its entirety. Said ASCII copy, created on Dec. 20, 2012, is named SC140UUS.txt and is 1,185 bytes in size.

### FIELD OF INVENTION

The present invention relates generally to agents that modulate the expression or activity of the PLOD-2 enzyme. Methods for improving the aesthetic appearance and health of human skin and also methods for identifying compounds useful for treating skin are provided. In particular, the invention relates to compounds that stimulate PLOD-2 expression in skin.

### BACKGROUND

Collagen is the body's major structural protein. It is composed of three protein chains wound together in a tight triple helix to form fibrils. The fibrils are cross-linked in the extracellular matrix to provide the structural scaffolding surrounding cells that helps to support cell shape and differentiation. The mesh-like collagen network binds cells together and provides the supportive framework or environment in which cells develop and function. The stimulation of collagen gives the skin its strength, durability, and smooth, plump appearance.

N-Acetyl-Tyrosinamide is an amino acid derivative with potent anti-aging and cosmetic benefits, as described in U.S. Pat. RE 41,278 and U.S. Pat. RE 41,339, the disclosures of which are hereby incorporated by reference. The present inventors have investigated the mode of operation of N-Acetyl-Tyrosinamide and discovered that it is a potent stimulator of the enzyme PLOD-2 (procollagen-lysine, 2-oxoglutarate 5-dioxygenase 2 (lysine hydroxylase-2), a homodimeric enzyme that is critical in the collagen maturation process.

Collagen synthesis and maturation is a complex multistep process. The PLOD-2 enzyme plays an important role in the collagen maturation process; it catalyzes the hydroxylation of lysine residues in the nascent procollagen protein strands. The resultant hydroxylysyl groups aid in the formation of the triple helix and serve as attachment sites for cross linking in the extracellular matrix. See, Van der Slot et al., 2003, J. Biol. Chem., 278:40967-40972; Walker et al., 2005, Matrix Biology, 23:515-523; Wu et al., 2006, Exp. Cell Res., 312:3485-3494. Thus, this modification is important for the stability of procollagen, the intermolecular cross linking of collagen fibrils and ultimately the maintenance of the dermal matrix.

The foregoing discussion is presented solely to provide a better understanding of nature of the problems confronting the art and should not be construed in any way as an admission as to prior art.

### SUMMARY OF THE INVENTION

In accordance with the foregoing objectives and others, the present invention provides modulators of the PLOD-2 enzyme, along with cosmetic and pharmaceutical compositions and methods for improving one or more signs of dermatological aging. PLOD-2 modulators encompassed by the instant invention are believed to upregulate levels of PLOD-2 in the skin. It is believed that upregulators of PLOD-2 within dermal cells (fibroblasts and/or keratinocytes) lead to increased collagen production. Given the importance of collagen to overall skin strength and health, upregulation of PLOD-2 will have a beneficial effect on reducing the appearance of aging on skin.

In one aspect of the invention, cosmetic compositions are provided for improving the aesthetic appearance of human skin comprising a cosmetically acceptable vehicle, and an effective amount of an active agent that modulates PLOD-2. In some embodiments, PLOD-2 will be increased by at least 25% when cells are incubated in a 0.05% solution of a PLOD-23 modulator. (See Example 3.) In some embodiments, the active agent is not N-Acetyl-Tyrosinamide, in other embodiments, the active agent will include N-Acetyl-Tyrosinamide in addition to a second PLOD-2 modulator.

In another aspect of the invention, a method is provided for improving the aesthetic appearance of human skin comprising topically applying to an area of the skin in need thereof (e.g., wrinkled skin) an effective amount of an active agent that stimulates PLOD-2 expression by at least 25% when measured after contact with a 0.05% solution of a PLOD-2 modulator, for a time sufficient to improve the aesthetic appearance of said human skin. In some, but not all embodiments, the active agent does not comprise N-Acetyl-Tyrosinamide. In all embodiments, N-acetyl-tyrosidamide is not included as a sole PLOD-2 modulator.

In another aspect of the invention, a method is provided for screening candidate substances to identify actives useful for improving the aesthetic appearance of skin. The method comprises assaying candidate substances for their ability to modulate PLOD-2 in a skin cell. In one embodiment, the method comprises assaying candidate substances for ability to modulate PLOD-2 levels in skin cells. PLOD-2 modulation may manifest as a modulation in gene transcription, translation, post-translational modification, or otherwise. The method typically involves incubating dermal fibroblasts or keratinocytes with a candidate substance and subsequently measuring the levels of mRNA encoding PLOD-2. The assay may measure the levels of any homolog, fragment or marker of PLOD-2, but typically what is measured are the expression levels of PLOD-2, which is expressed by the human gene PLOD-2. The expression levels of the human gene PLOD-2 may be determined, for example, by measuring the expression levels of the corresponding mRNA by any suitable technique, such as quantitative polymerase chain reaction (qPCR). The cell used in the assay may be any animal cell that expresses PLOD-2, but is typically a human cell.

In another aspect of the invention, a method is provided for screening active agents useful for improving the aesthetic appearance of skin (e.g., reducing wrinkles) comprising assaying candidate substances for their ability to modulate PLOD-2 expression in a human dermal fibroblast or keratinocytes, wherein an change in mRNA encoding PLOD-2 is indicative of a PLOD-2 modulator.

Methods are also provided for improving the aesthetic appearance of human skin comprising topically applying to an area of the skin in need thereof a composition comprising a retinoid and an effective amount of an active agent that modulates cellular levels of PLOD-2. The PLOD-2 modulators are referred to herein as “actives”, and typically will be formulated in a cosmetically acceptable vehicle and topically applied to a human integument, such as the skin of the face, neck, lips, hands, chest, legs, etc., for a time sufficient to enhance the health or aesthetic appearance thereof, including reducing the number or severity of wrinkles and/or fine lines.

In another aspect of the invention, a method is provided of treating wrinkles and/or fines lines comprising topically applying to an area of skin in need thereof an effective amount of N-Acetyl-Tyrosinamide and a second active agent that stimulates PLOD-2.

In one aspect of the invention, a method is provided for improving the aesthetic appearance of human skin comprising topically applying to an area of the skin in need thereof an effective amount of an active agent that stimulates (e.g., upregulates) cellular levels of PLOD-2, wherein the ability of the active agent to modulate PLOD-2 has been determined by an assay which measures the expression level of PLOD-2 in a cell that has been contacted with the active agent. The cell used in the assay may be any animal cell that expresses PLOD-2, but is typically a human cell. The cell may be a skin cell, such as a fibroblast or keratinocyte.

A method of treating (i.e., reducing the number or severity or depth) wrinkles and/or fines lines is also provided, comprising topically applying to an area of the skin in need thereof an effective amount of an active agent that modulates PLOD-2, alone or in combination with a second active (e.g. a retinoid (e.g., retinol)). The ability of the active agent to modulate PLOD-2 may be determined by the inventive assay which measures the expression level of PLOD-2 in a cell that has been contacted with the active agent.

In yet another aspect of the invention, methods are provided for improving the aesthetic appearance of human skin comprising topically applying to an area of the skin in need thereof an effective amount (e.g., 0.01%-5% by weight, w/w) of retinol in combination with an effective amount (e.g., 0.001%-5% by weight, w/w) of an active agent that modulates cellular levels of PLOD-2.

Further aspects, features and advantages of the present invention will be better appreciated upon a reading of the detailed description of the invention.

### DETAILED DESCRIPTION OF THE INVENTION

Detailed embodiments of the present invention are disclosed herein; however, it is to be understood that the disclosed embodiments are merely illustrative of the invention that may be embodied in various forms. In addition, each of the examples given in connection with the various embodiments of the invention are intended to be illustrative, and not restrictive. Therefore, specific structural and functional details disclosed herein are not to be interpreted as limiting, but merely as a representative basis for teaching one skilled in the art to variously employ the present invention.

All terms used herein are intended to have their ordinary meaning unless otherwise provided. By “cosmetically acceptable,” it is meant that a particular component is generally regarding as safe and non-toxic at the levels employed. The term “prevent,” as used herein, includes delaying the onset of or progression of a particular sign of skin aging. The term “thin skin” includes skin that becomes thinner with chronological aging as well as prematurely thinned skin, which may be caused, for example, by photo-aging. In one embodiment, the prematurely thinned skin has been diagnosed as such by a clinician. The phrase “individual in need thereof” refers to a human that could benefit from improved dermal appearance or health, including males or females, typically females. The term “skin” includes, without limitation, the lips, skin of the face, hands, arms, neck, scalp, and chest. As used herein, the term “consisting essentially of” is intended to limit the invention to the specified materials or steps and those that do not materially affect the basic and novel characteristics of the claimed invention, as understood from a reading of this specification.

As used herein, the term “PLOD-2” refers to the enzyme procollagen-lysine, 2-oxoglutarate 5-dioxygenase 2 (lysine hydroxylase-2), in all of its various isoforms, from an organism including without limitation human, mouse, or other animal. In some embodiments, the PLOD-2 enzyme is human PLOD-2, including without limitation Isoform CRA_a (Accession No. EAW78939.1; see also Isoform 2 Precursor, Accession No. NP_{—}891988.1), Isoform CRA_b (Accession No. EAW78940.1), and Isoform CRA_c (Accession No. EAW78941.1; see also Isoform 1 Precursor, Accession No. NP_{—}000926.2). The nomenclature used herein to describe specific PLOD-2 examples is that of the National Center for Biotechnology Information (“NCBI”), Accession Numbers EAW78939.1 (Isoform CRA_a), NP_{—}891988.1 (Isoform 2 Precursor), EAW78940.1 (Isoform CRA_b), EAW78941.1 (Isoform CRA_c), and NP_{—}000926.2 (Isoform 1 Precursor), which are hereby incorporated by reference.

The term “modulator” encompasses any substance, including, without limitation, organic molecules; biomolecules (e.g., peptides, proteins, antibodies, nucleic acid oligomers, etc.); and combinations of substances, such as botanical extracts. The stimulators may stimulate the cellular levels of the PLOD-2 enzyme, by which is meant that the cellular levels of PLOD-2 protein are increased by the active agent. The term “smodulation” may refer to upregulation, downregulation, induction, stimulation, and/or potentiation, or relief of inhibition. The stimulators may also be, without limitation, activators, inhibitors, agonists, and/or antagonists, which are compounds that, for example, bind to, stimulate, increase, decrease, open, close, activate, deactivate, facilitate, enhance activation, decrease activation, sensitize, desensitize, upregulate, or downregulate expression levels of PLOD-2. The mechanism by which the protein level is modulated is not important.

As used herein, the term “expression levels” refers to an amount of a gene and/or protein that is expressed in a cell. As used herein, a “gene” includes a polynucleotide containing at least one open reading frame that is capable of encoding a particular polypeptide. As used herein, the terms “polynucleotide” is synonymous with “oligonucleotide” and includes polymeric forms of nucleotides of any length, either deoxyribonucleotides or ribonucleotides, or analogs thereof, including, without limitation, mRNA, DNA, cDNA, primers, probes, and the like.

The discovery of the correlation between the expression levels of PLOD-2 and dermatological aging has led to a screening method for identifying potential PLOD-2 stimulators. In one embodiment, an assay is provided for determining the expression levels of PLOD-2 after a cell has been treated, incubated, or otherwise contacted with a candidate substance. The term “candidate substance” refers to any substance that is tested for activity as a modulator of PLOD-2, whether or not the substance is suspected of possessing such activity, other than N-Acetyl-Tyrosinamide as a sole PLOD-2 modulator. The cell can be any cell from any animal that expresses PLOD-2. In one embodiment, the cell is a dermal fibroblast. In another embodiment, the cell is a keratinocyte. The cell may be a human or mouse cell. After the cell has been incubated with a candidate substance for a sufficient length of time to provide a measurable change in expression levels, which will typically be at least one hour, and more typically from about 12 hours to about 72 hours, the cell is then lysed to release the cellular components, such as PLOD-2 and mRNA encoding PLOD-2. The amount of PLOD-2 protein or mRNA may then be measured by any suitable technique for detection and quantitation of peptides and proteins and/or polynucleotides (e.g., mRNA).

In some embodiments, the methods for measuring expression levels of PLOD-2 involve the quantitation of mRNA expression. Suitable methods for determining mRNA expression include quantitative PCR (QPCR), real-time QPCR, reverse transcription PCR (RT-PCR), and quantitative reverse transcription PCR (QRT-PCR), as are well-known in the art. As described in detail in U.S. Pat. Nos. 7,101,663 and 7,662,561, the disclosures of which are hereby incorporated by reference, a quantitative reverse transcriptase polymerase chain reaction (QRT-PCR) for detecting mRNA may include the steps of: (a) incubating an RNA sample from the cellular lysate with a reverse transcriptase and a high concentration of a target sequence-specific reverse transcriptase primer under conditions suitable to generate cDNA; (b) subsequently adding suitable polymerase chain reaction (PCR) reagents to the reverse transcriptase reaction, including a high concentration of a PCR primer set specific to the cDNA and a thermostable DNA polymerase to the reverse transcriptase reaction; and (c) cycling the PCR reaction for a desired number of cycles and under suitable conditions to generate PCR products (“amplicons”) specific to the cDNA. The products of the QRT-PCR process may be compared after a fixed number of PCR cycles to determine the relative quantity of the RNA species as compared to a given reporter gene, for example, by Southern blotting. More typically, the progress of the PCR reaction is monitored by analyzing the relative rates of amplicon production for each PCR primer set, for example, by (1) non-specific fluorescent dyes that intercalate with any double-stranded DNA, and/or (2) sequence-specific DNA probes consisting of oligonucleotides that are labeled with a fluorescent reporter which permits detection only after hybridization of the probe with its complementary DNA target.

The mRNA may be any mRNA that is associated with PLOD-2 from any animal, including the mRNAs encoding PLOD-2, or any polynucleotide, fragment or derivative thereof. In one embodiment, the mRNA encodes human PLOD-2.

The level of mRNA expression may be compared to controls that are not treated with the candidate substance to determine the relative degree of stimulation. In some embodiments, the candidate substance will upregulate or downgrade mRNA expression by at least about 25%, more suitably at least about 40%, at least about 50%, at least about 60%, or at least about 70%. The candidate substance may, for example, upregulate or downregulate mRNA expression by at least about 60%, at least about 70%, at least about 80%, at least about 90%, or at least about 100%. The evaluation of the % upregulation or downregulation may be made according to the protocol set forth in Example 3, and in particular, may be measured by exposing cells to an 0.5% solution of the PLOD-2 modulator. Where the active is a mixture of compounds, such as a botanical extract, the measurement may be made at a 0.05% by weight, for example. Candidate substances meeting these criteria may be selected for use of for further evaluation.

The cosmetic compositions of this invention may further comprise a retinoid and an effective amount of an active agent that modulates cellular levels of PLOD-2. Retinoids may be without limitation retinol (Vitamin A) and esters thereof, such as retinol palmitate, retinol acetate and retinol propionate, and salts thereof, retinaldehyde, or retinoic acid (e.g., all-trans or 13-cis) and derivatives thereof. The cosmetic compositions of this invention may further comprise alpha-hydroxy acids, such as glycolic acid.

The PLOD-2 modulators of the invention, such as those identified by the foregoing screening protocol, may be used as active agents in cosmetic preparation and may be formulated with other cosmetically acceptable components, and vehicles, into a composition for topical application to the skin. The compositions are topically applied to the skin in effective amounts, by which is meant an amount sufficient to achieve a measurable improvement in skin health or reduction in one or more dermatological signs of aging with daily (once, twice, etc.) administration, typically for a period of at least one week or more. Such signs of skin aging include without limitation, the following:

(a) treatment, reduction, and/or prevention of fine lines or wrinkles;

(b) reduction of skin pore size;

(c) improvement in skin thickness, plumpness, and/or tautness;

(d) improvement in skin smoothness, suppleness and/or softness;

(e) improvement in skin tone, radiance, and/or clarity;

(f) improvement in procollagen, and/or collagen production;

(g) improvement in maintenance and remodeling of elastin;

(h) improvement in skin texture and/or promotion of retexturization;

(i) improvement in skin barrier repair and/or function;

(j) improvement in appearance of skin contours;

(k) restoration of skin luster and/or brightness;

(l) replenishment of essential nutrients and/or constituents in the skin;

(m) improvement of skin appearance decreased by aging and/or menopause;

(n) improvement in skin moisturization;

(o) increase in skin elasticity and/or resiliency;

(p) treatment, reduction, and/or prevention of skin sagging;

(q) improvement in skin firmness; and/or

(r) reduction of pigment spots and/or mottled skin; and

(s) improvement of optical properties of skin by light diffraction or reflection.

In practice, the compositions of the invention, including agents that modulate PLOD-2 expression levels, alone, or in cosmetically acceptable vehicles, are applied to skin in need of treatment. That is, skin which suffers from a deficiency or loss in any of the foregoing attributes or which would otherwise benefit from improvement in any of the foregoing skin attributes. The skin is typically treated once or twice daily. The treatment may continue for a week, two weeks, four weeks, eight weeks, six months or longer.

In one embodiment the active agents are topically applied, in a cosmetically acceptable vehicle, to skin suffering from fine lines and/or wrinkles to prevent, treat, and/or amelioration the appearance of the fine lines and/or wrinkles in the skin. In this case, the compositions are applied to skin in need of treatment, by which is meant skin already having wrinkles and/or fine lines or skin that is at risk of developing fine lines and/or wrinkles. The compositions may be applied directly to the fine lines and/or wrinkles on the skin of the face, neck, lips, chest, and/or hands. Upregulators of PLOD-2 expression can remediate signs of aging by enhancing production of collagen in skin.

In one embodiment, the invention is directed to a method of improving the aesthetic appearance of skin by increasing the production of collagen in the skin, the method comprising topically applying to an area of the skin in need thereof an effective amount of an agent that upregulates PLOD-2 expression.

In one embodiment, the compound is not an amino acid or a derivative thereof. In another embodiment, the compound is not an N-acetyl derivative of an amino acid and/or an amide derivative of an amino acid, as described in U.S. Pat. RE 41,278 or U.S. Pat. RE 41,339, the disclosures of which are hereby incorporated by reference. In a further embodiment the compound is not an N-acetyl-aldosamine or a derivative thereof, as described in U.S. Pat. RE 41,278 or U.S. Pat. RE 41,339. In another embodiment, the compound is not an N-acetylamino acid or a derivative thereof, as described in U.S. Pat. RE 41,278 or U.S. Pat. RE 41,339. In another embodiment, the PLOD-2 stimulator is not Coenzyme Q10. In some embodiments, each of the compounds described in U.S. Pat. RE 41,278 or U.S. Pat. RE 41,339 are hereby incorporated by reference and explicitly excluded from the compositions and treatment methods of the invention.

In one embodiment, the PLOD-2 stimulators will have a structure according to Formula I(a):

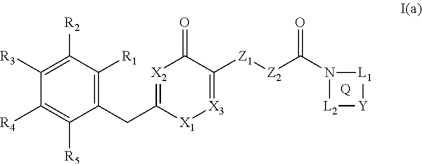

where Q represents a 3-6 membered heterocyclic ring, wherein L₁ and L₂ are independently selected from a bond or a group E₁-(L₃)ₗ-ε₂, where “l” is an integer from 0 to 4 (including 0, 1, 2, 3, and 4); where L₃ is selected from —CH₂—, —CHR*—, —C(R*)₂—, —CH—, —CR*—, or a bond; and ε₁ and ε₂ are independently selected from —N—, —NH—, —NR^{N}—, —O—, —S—, —(C═O)—, or a bond; and in one embodiment, L₃ is —CH₂— and l is an integer 1, 2, or 3, including an embodiment where L₃ is CH₂ where l is 2 and ε₁ and ε₂ are both a bond (i.e., absent) such that the groups L₁ and/or L₂ have the form CH₂—CH₂—; and in a particular embodiment, L₁ and L₂ both have the form CH₂CH₂, although any of the hydrogens in the ethylene radical may be optionally substituted with groups R (e.g., with methyl, etc.);

Y is selected from —CH—, —CR*—, —CHR*—, —C(R*)₂—, —N—, —O—, —S—, —NR^{N}—, or a bond (i.e., absent); but is typically —CH— or —CR*—, where R* is as defined above, with special mention being made of lower alkyl (i.e., methyl, ethyl, propyl, isopropyl, butyl, etc.).

Z₁ represents a bond, —CH₂—, or (C═O)—; and is typically (but not necessarily) —CH₂—; Z₂ represents a bond, —O—, —NH—, —NR^{N}—, —CH₂—, —CHR*—, —C(R*)₂—, —(CH₂)ₚ—, —(CH₂)ₚ—NH, or —(CH₂)ₚ—NR^{N}—, where p is an integer from 1-6; and is typically (but not necessarily) —CH₂—. In one embodiment, Z₁ and Z₂ together form —CH₂—CH₂—, although any of the hydrogens in the ethylene radical may be optionally substituted with groups R (e.g., with methyl, etc.);

X₁ represents —CH₂—, —CHR*—, —C(R*)₂—, —NH—, —NR^{N}—, —O—, or —S—; and in some embodiments, X₁ represents NH or NR^{N}, where R^{N} is as defined above, with special mention being made of lower alkyl (i.e., methyl, ethyl, propyl, isopropyl, butyl, etc.). X₂ and X₃ may be independently selected from nitrogen atoms, —CH—, and —CR*—. In one embodiment, X₂ and/or X₃ are nitrogen atoms.

R₁-R₅ are independently selected, at each occurrence, from a group R; wherein R is selected from hydrogen, —F; —Cl; —Br; —I; —OH, —OR*; —NH₂; —NHR*; —N(R*)₂; —N(R*)₃⁺; —N(R*)—OH; —N(→O)(R*)₂; —O—N(R*)₂; —N(R*)—O—R*; —N(R*)—N(R*)₂; —C═NR*; —N═C(R*)₂; C═N—N(R*)₂; —C(═NR*)—N(R*)₂; —SH; —SR*; —CN; —NC; —(C═O)R*; —CHO; —CO₂H; —CO₂⁻; —CO₂R*; —(C═O)—S—R*; —O—(C═O)—H; —O—(C═O)—R*; —S—(C═O)—R*; —(C═O)—NH₂; —(C═O)—N(R*)₂; —(C═O)—NHNH₂;—O—(C═O)—NHNH₂; —(C═S)—NH₂; —(C═S)—N(R*)₂; —N(R*)—CHO; —N(R*)—(C═O)R*; —(C═NR)—O—R*; —O—(C═NR*)—R*, —SCN; —NCS; —NSO; —SSR*; —N(R*)C(═O)—N(R*)₂; —N(R*)—C(═S)—N(R*)₂; —SO₂R*; —O—S(═O)₂—R*; —S(═O)₂—OR*; —N(R*)—SO₂—R*; —SO₂—N(R*)₂; —O—SO₃⁻; —O—S(═O)₂—OR*; —O—S(═O)OR*; —O—S(═O)R*; —S(═O)OR*; —S(═O)—R*; —NO; —NO₂; —NO₃; —O—NO; —O—NO₂; —N₃; —N₂—R*; —N(C₂H₄); —Si(R*)₃; —CF₃; —O—CF₃; —PR*₂; —O—P(═O)(OR*)₂; —P(═O)(OR*)₂; perfluoroalkyl; an aliphatic C₁-C₁₂ hydrocarbon radical; a C₁-C₁₂ aromatic hydrocarbon radical; or a C₁-C₁₂ heteroaryl radical; where R* is independently at each occurrence hydrogen or a straight chained, branched, or cyclic C₁-C₂₀ hydrocarbon radical, which may be saturated, partially saturated, or aromatic, each of which may be optionally substituted with 1-6 heteroatoms selected from nitrogen, oxygen, sulfur, or halogen;

R^{N} is hydrogen or a straight chained, branched, or cyclic saturated, partially saturated, or aromatic C₁-C₂₀ hydrocarbon radical, optionally substituted with 1-6 heteroatoms selected from nitrogen, oxygen, sulfur, or halogen; and cosmetically acceptable salts of the compounds of Formula I(a).

In some embodiments of the compounds of Formula I(a), L₁ is —(CH₂)ₘ—, and/or L₂ is —(CH₂)ₙ—, wherein “m” and “n” are independently selected from integers from 1-3; and Y is selected from —N—, —NR^{N}—, —S—, —O—, or a bond. In other embodiments, Q defines a heterocycle selected from the group consisting of aziridine, diaziridine, oxaziridine, azetidine, diazetidine, oxazetidine, pyrrolidine, oxazolidine, thiazolidine, imidazolidine, pyrazolidine, pyrrole, imidazole, pyrazole, 1,3,4-triazole, 1,2,3-triazole, piperidine, 4-alkyl-piperidine, morpholino, piperazine, and thiomorpholine, each being optionally substituted with one or more groups R. For example, Q may be a heterocycle selected from the group consisting of pyrrolidine, piperidine, morpholino, and piperazine, each being optionally substituted with one or more groups R (e.g., methyl). In one illustrative embodiment, ring Q is 4-methyl-piperidine.

In some embodiments of the compounds of Formula I(a), Z₁ is a bond (i.e., it is altogether absent) or —CH₂—; and Z₂ is a bond, —NH— or —CH₂—. In other embodiments of the compounds of Formula I(a), Z₁ is —CH₂—; and Z₂ is —CH₂—. In other embodiments of the compounds of Formula I(a), X₁ may be —NH— or —NR^{N}; and/or X₂ and/or X₃ may be nitrogen. Typically (but not necessarily) R₁, R₄, and/or R₅ are hydrogen; and R₂ and/or R₃ may be a group —OR*, where R* is C₁₋₆ alkyl. In one implementation, R₂ and/or R₃ are a group —OCH₃. In one illustrative embodiment, Z₁ is —CH₂; Z₂ is —CH₂—; X₁ is —NH—; and X₂ and X₃ are each nitrogen.

The invention embraces the use of cosmetically or pharmaceutically acceptable (e.g., non-toxic and/or non-irritating) salts of the compounds of Formula I(a). Examples of the salts of the compounds in the present invention include salts with alkali metals such as sodium and potassium; salts with alkaline-earth metals such as calcium and magnesium; salts with amines such as monoethanolamine; salts with inorganic acids such as hydrochloric acid and sulfuric acid; and salts with organic acids such as citric acid and acetic acid. Special mention may be made of acid addition salts and in particular hydrochloride salts.

In one embodiment of the invention, the PLOD-2 stimulators comprise a compound (or salt thereof) having the structure of Formula I(b):

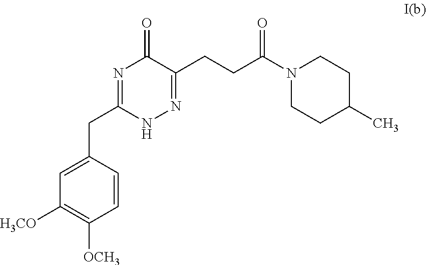 [SMILES: COc1ccc(Cc2nc(=O)c(CCC(=O)N3CCC(C)CC3)n[nH]2)cc1OC]

The compound of Formula I(b) is synonymously identified as ALB-H09784893 herein, which in one embodiment a species of which may be designated as CAS #107720-70-6. ALB-H09784893 is a potent stimulator of PLOD-2.

In another embodiment, the compounds of the invention may have a structure according to formula II(a):

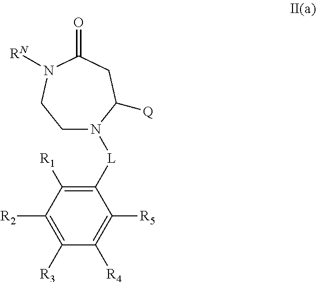

wherein, L is a bond (i.e., L is absent) or a divalent radical selected from —(C═O), —O—(C═O), —N(R*)—(C═O)—, —(CH₂)ₖ—, —O—(CH₂)ₖ—, —N(R*)—(CH₂)ₖ—, —(CH₂)ₖ—(C═O)—, —(CH₂)ₖ—O—(C═O)—, —(CH₂)ₖ—N(R*)—(C═O), where K is an integer from 1-3, and wherein L is typically —CH₂—;

Q represents a five-membered heterocyclic ring selected from the following:

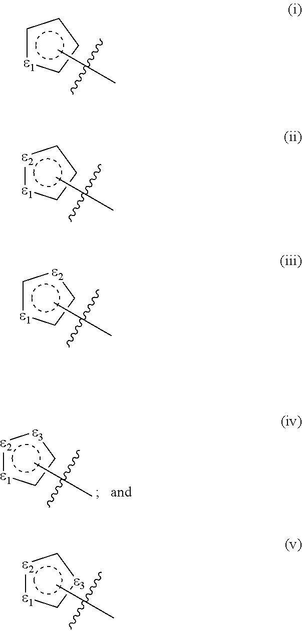

wherein ε₁, ε₂, and ε₃, are independently selected from N, NH, NR*, S, and O; with the proviso that where the point of attachment is ε₁, ε₂, or ε₃, then that position represents N; and wherein carbon atoms which are not the point of attachment may be optionally substituted with a group R; and wherein the dashed circles indicate that each ring may comprise zero, one, or two double bonds, although Q is typically a heteroaryl moiety;

R₁-R₅ are independently selected, at each occurrence, from a group R; wherein R is selected from hydrogen, —F; —Cl; —Br; —I; —OH, —OR*; —NH₂; —NHR*; —N(R*)₂; —N(R*)₃⁺; —N(R*)—OH; —N(→O)—(R*)₂; —O—N(R*)₂; —N(R*)—O—R*; —N(R*)—N(R*)₂; —C═N—R*; —N═C(R*)₂; —C═N—N(R*)₂; —C(═NR*)—N(R*)₂; —SH; —SR*; —CN; —NC; —(C═O)R*; —CHO; —CO₂H; —CO₂⁻; —CO₂R*; —(C═O)S—R*; —O—(C═O)H; —O—(C═O)—R*; —S(C═O)—R*; —(C═O)—NH₂; —(C═O)—N(R*)₂; —(C═O)—NHNH₂; —O—(C═O)NHNH₂; —(C═S)NH₂; —(C═S)—N(R*)₂; —N(R*)—CHO; —N(R*)—(C═O)—R*; —(C═NR)—O—R*; —O—(C═NR*)—R*, —SCN; —NCS; —NSO; —SSR*; —N(R*)—C(═O)—N(R*)₂; —N(R*)—C(═S)—N(R*)₂; —SO₂—R*; —O—S(═O)₂R*; —S(═O)₂—OR*; —N(R*)—SO₂—R*; —SO₂—N(R*)₂; —O—SO₃⁻; —O—S(═O)₂—OR*; —O—S(═O)—OR*; —O—S(═O)—R*; —S(═O)—OR*; —S(═O)—R*; —NO; —NO₂; —NO₃; —O—NO; —O—NO₂; —N₃; —N₂—R*; —N(C₂H₄); —Si(R*)₃; —CF₃; —O—CF₃; —PR*₂; —O—P(═O)(OR*)₂; —P(═O)(OR*)₂; perfluoroalkyl; vinyl, allyl, an aliphatic C₁-C₁₂ hydrocarbon radical (e.g., methyl, ethyl, propyl, isopropyl, etc.); a C₅-C₁₂ aromatic hydrocarbon radical (e.g., phenyl); or a C₅-C₁₂ heteroaryl radical; where R* is independently at each occurrence hydrogen or a branched, cyclic, or straight chained C₁-C₂₀ hydrocarbon radical that is saturated, partially saturated, or aromatic, optionally substituted with 1-6 heteroatoms selected from nitrogen, oxygen, sulfur, or halogen;

Typically, one of R₁, R₂, R₃, R₄, and/or R₅ is —CN and at least one of the remaining substituents R₁, R₂, R₃, R₄, and/or R₅ may be hydrogen; more typically one of R₁, R₂, R₃, R₄, and/or R₅ is —CN, and in one embodiment R₂ is —CN, and all of the remaining substituents R₁, R₂, R₃, R₄, and/or R₅ are hydrogen.

R^{N} is hydrogen or a saturated, partially saturated, or aromatic C₁-C₂₀ hydrocarbon radical, optionally substituted with 1-6 heteroatoms selected from nitrogen, oxygen, sulfur, or halogen, R^{N} is typically hydrogen, methyl, or ethyl, but is typically hydrogen or methyl.

The invention embraces the use of cosmetically or pharmaceutically acceptable (e.g., non-toxic and/or non-irritating) salts of the compounds of Formula II(a). Examples of the salts of the compounds in the present invention include salts with alkali metals such as sodium and potassium; salts with alkaline-earth metals such as calcium and magnesium; salts with amines such as monoethanolamine; salts with inorganic acids such as hydrochloric acid and sulfuric acid; and salts with organic acids such as citric acid and acetic acid. Special mention may be made of acid addition salts and in particular hydrochloride salts.

In some embodiments, Q is a 5-membered nitrogen-containing heteroaryl group selected from:

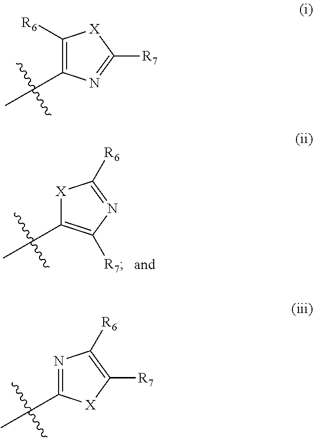

where X is either an oxygen atom, a sulfur atom, or NR*, and R₆ and R₇ are independently selected from groups R as defined above (although they are typically hydrogen). In some embodiments, Q is a group:

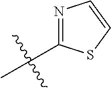 [SMILES: *c1nccs1]

In some embodiment of the compounds according to Formula II(a), the substituent R^{N} is hydrogen or methyl (typically hydrogen) and/or one of (and typically all of) R₁, R₃, R₄, or R₅ are hydrogen. In one embodiment R₂ is —CN.

In another aspect of the invention, cosmetic compositions are provided comprising a compound (or salt thereof) having the structure II(b):

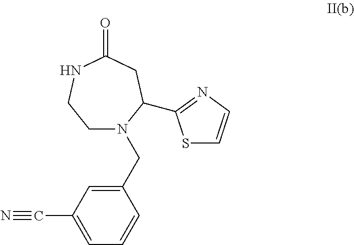 [SMILES: N#Cc1cccc(CN2CCNC(=O)CC2c2nccs2)c1]

The compound of Formula II(B) is identified herein as ALB-H16063163. ALB-H16063163 is a potent stimulator of PLOD-2.

The compounds of Formula I(a), I(b), II(a), and II(b) may be racemic or may comprise an enantiomeric excess of either the R or S enantiomer. In some embodiments, the compound comprise an enantiomeric excess of at least about 90% of the R enantiomer; in other embodiments the compound comprises an enantiomeric excess of at least about 90% of the S enantiomer.

The PLOD-2 stimulators or modulators, including the compounds of Formulas I(a), I(b), II(a), and II(b), may be formulated in cosmetically acceptable vehicles, which may comprises one or more of a film forming polymer, a thickener, a pH adjuster, a preservative, an emulsifier, a gelling agent, an antioxidant, a fragrance, a colorant, and the like. The vehicle may comprise a water-in-oil, oil-in-water, silicone-in-water, or water-in-silicone emulsion and will typically further comprise an emulsifier. The formulations may optionally include a retinoid, for example retinoic acid, retinol, retinal, retinyl acetate, fatty acid ester of retinol, such as retinyl palmitate, to name a few.

The cosmetic compositions according to the invention can be formulated in a variety of forms for topical application and will comprise from about 0.00001% to about 90% by weight of one or more actives that modulate (e.g., stimulate) PLOD-2, and typically will comprise such actives in an amount from about 0.0001% to about 25% by weight, and more typically from about 0.001% to about 10% by weight. In some embodiments, the PLOD-2 stimulators will individually or collectively comprise from 0.01% to about 25% by weight of the composition. When the cosmetic compositions according to the invention are formulated in a liquid form, they will typically comprise from about 0.001 μM to about 50 μM of one or more actives that modulate PLOD-2, and more typically will comprise such actives in an amount from about 0.5 μM to about 10 μM, or from about 2.25 μM to about 10 μM. When reference is made herein to % stimulation of PLOD-2, unless otherwise stated, such activity is determined utilizing treatment of cells with a ______% solution of a PLOD-2 modulator and assaying subsequent cellular response, in accordance with the procedure of Example 3.

In some embodiments, the active agents upregulate PLOD-2 by at least 25%, 40%, 50%, 60%, 70%, 80%, or more, when measured at a concentration of about 2.25 μM (or about 0.05% by weight, in the case of botanical extracts), of a stimulator of PLOD-2. In other embodiments, the active agents downregulate PLOD-2 by at least 30%, 40%, 50%, 60%, 70%, 80%, or more, when measured at a concentration of about 2.25 μM (or about 0.05% by weight, in the case of botanical extracts), of a stimulator of PLOD-2.

The compositions can include a cosmetically acceptable vehicle. Such vehicles may take the form of any known in the art suitable for application to skin and may include, but are not limited to, water; vegetable oils; mineral oils; esters such as octal palmitate, isopropyl myristate and isopropyl palmitate; ethers such as dicapryl ether and dimethyl isosorbide; alcohols such as ethanol and isopropanol; fatty alcohols such as cetyl alcohol, cetearyl alcohol, stearyl alcohol and biphenyl alcohol; isoparaffins such as isooctane, isododecane and is hexadecane; silicone oils such as cyclomethicone, hydrocarbon oils such as mineral oil, petrolatum, isoeicosane and polyisobutene; polyols such as propylene glycol, glycerin, butylene glycol, pentylene glycol and hexylene glycol; liposomes; waxes; or any combinations or mixtures of the foregoing.

The vehicle may comprise an aqueous phase, an oil phase, an alcohol, a silicone phase or mixtures thereof and may be in the form of an emulsion. Non-limiting examples of suitable emulsions include water-in-oil emulsions, oil-in-water emulsions, silicone-in-water emulsions, water-in-silicone emulsions, polyol-in-oil emulsions, oil-in-polyol, polyol-in-silicone, and silicone-in-polyol emulsions, wax-in-water emulsions, water-oil-water triple emulsions or the like. The emulsion may include an emulsifier (e.g., 0.01% to 10% by weight), such as a nonionic, anionic or amphoteric surfactant, or a gelling agent.

The topical composition will typically have a pH range from 1 to 8, with a pH in the range of from 2 to 7 being typical. In some embodiments, the composition will have a pH in the range of from 3.5 to 5.5. In some embodiments, the pH of the composition may be kept substantially below pH 4.0 so as to increase stability of a retinoid component. Suitable pH adjusters and/or chelators such as citric acid, ascorbic acid, EDTA, and/or triethanolamine may be added to bring the pH within the desired range.

In one embodiment of the invention, the compositions may include additional skin actives, including but not limited to, retinoids, botanicals, keratolytic agents, desquamating agents, keratinocyte proliferation enhancers, collagenase inhibitors, elastase inhibitors, depigmenting agents, anti-inflammatory agents, steroids, anti-acne agents, antioxidants, and advanced glycation end-product (AGE) inhibitors.

The composition may comprise additional active ingredients having anti-aging benefits, as it is contemplated that synergistic improvements may be obtained with such combinations. Exemplary anti-aging components include, without limitation, botanicals (e.g., Butea frondosa extract); phytol; thiodipropionic acid (TDPA) and esters thereof; retinoids (e.g., 9-cis retinoic acid, 13-cis retinoic acid, all-trans retinoic acid and derivatives thereof, phytanic acid, retinol (Vitamin A) and esters thereof, such as retinol palmitate, retinol acetate and retinol propionate, and salts thereof and others); hydroxy acids (including alpha-hydroxy acids and beta-hydroxy acids), salicylic acid and alkyl salicylates; exfoliating agents (e.g., glycolic acid, 3,6,9-trioxaundecanedioic acid, etc.), estrogen synthetase stimulating compounds (e.g., caffeine and derivatives); compounds capable of inhibiting 5 alpha-reductase activity (e.g., linolenic acid, linoleic acid, finasteride, and mixtures thereof); and barrier function enhancing agents (e.g., ceramides, glycerides, cholesterol and its esters, alpha-hydroxy and omega-hydroxy fatty acids and esters thereof, etc.), to name a few.

Exemplary retinoids include, without limitation, retinoic acid (e.g., all-trans or 13-cis), and derivatives thereof, retinaldehyde, retinol (Vitamin A) and esters thereof, such as retinol palmitate, retinol acetate and retinol propionate, and salts thereof. It is contemplated that combinations of PLOD-2 modulators with any of these retinoids will provide enhanced or synergistic improvements to skin. The retinoids will typically be included in amounts from about 0.0001% to about 5% by weight, or from about 0.01% to about 2.5% by weight, or from about 0.1% to about 1.0% by weight. Compositions according to this embodiment will typically include antioxidants and/or chelators such as ascorbic acid, BHT, and/or disodium EDTA, alone or in combination.

In another embodiment, the topical compositions of the present invention may also include one or more of the following: a skin penetration enhancer; an emollient, such as isopropyl myristate, petrolatum, silicones (e.g., methicone, dimethicone), oils, mineral oils, and fatty acid esters; a humectant, such as glycerin or caprylyl glycol; a skin plumper, such as palmitoyl oligopeptide, collagen, or collagen and/or glycosaminoglycan (GAG) enhancing agents; a sunscreen, such as avobenzone; an exfoliating agent; and an antioxidant.

Suitable exfoliating agents include, for example, alpha-hydroxy acids, beta-hydroxy acids, oxa-acids, oxadiacids, and their derivatives such as esters, anhydrides and salts thereof. Suitable hydroxy acids include, for example, glycolic acid, lactic acid, malic acid, tartaric acid, citric acid, 2-hydroxyalkanoic acid, mandelic acid, salicylic acid and derivatives thereof. A notable exfoliating agent is glycolic acid. When present, the exfoliating agent may comprise from about 0.01% to about 20% by weight of the composition.

Examples of antioxidants that may be used in the present compositions include compounds having phenolic hydroxy functions, such as ascorbic acid and its derivatives/esters; beta-carotene; catechins; curcumin; ferulic acid derivatives (e.g., ethyl ferulate, sodium ferulate); gallic acid derivatives (e.g., propyl gallate); lycopene; reductic acid; rosmarinic acid; tannic acid; tetrahydrocurcumin; tocopherol and its derivatives; uric acid; or any mixtures thereof. Other suitable antioxidants are those that have one or more thiol functions (—SH), in either reduced or non-reduced form, such as glutathione, lipoic acid, thioglycolic acid, and other sulfhydryl compounds. The antioxidant may be inorganic, such as bisulfites, metabisulfites, sulfites, or other inorganic salts and acids containing sulfur. In one particular embodiment, the inventive compositions will include a combination of retinol, TDPA or an ester thereof, and a PLOD-2 modulator. Compositions of the present invention may comprise an antioxidant (e.g., from about 0.001 wt % to about 10 wt %, or from about 0.01 wt % to about 5 wt %, of the total weight of the composition).

Other conventional additives include: vitamins, such as tocopherol and ascorbic acid; vitamin derivatives such as ascorbyl monopalmitate; thickeners such as hydroxyalkyl cellulose; gelling agents; structuring agents; metal chelating agents such as EDTA or salts thereof; pigments; colorants; and pH adjusters. The composition may optionally comprise other components known to those skilled in the art including, but not limited to, film formers, moisturizers, minerals, viscosity and/or rheology modifiers, anti-acne agents, insect repellents, skin cooling compounds, skin protectants, lubricants, fragrances, preservatives, stabilizers, and mixtures thereof. In addition to the foregoing, the cosmetic compositions of the invention may contain any other compound for the treatment of skin disorders.

The composition may be formulated in a variety of product forms, such as, for example, an emulsion, lotion, cream, serum, spray, aerosol, cake, ointment, essence, gel, paste, patch, pencil, towelette, mask, stick, foam, elixir, concentrate, and the like, particularly for topical administration. Typically, the composition is formulated as an emulsion, lotion, cream, ointment, serum or gel.

The invention provides a method for treating aging skin by topically applying a composition comprising an active agent that stimulates PLOD-2, typically in a cosmetically acceptable vehicle, over the affected area for a period of time sufficient to remediate, reverse, reduce, ameliorate, or prevent dermatological signs of aging.

Generally, the improvement in the condition and/or aesthetic appearance is selected from the group consisting of: reducing dermatological signs of chronological aging, photo-aging, hormonal aging, and/or actinic aging; preventing and/or reducing the appearance of lines and/or wrinkles; reducing the noticeability of facial lines and wrinkles, facial wrinkles on the cheeks, forehead, perpendicular wrinkles between the eyes, horizontal wrinkles above the eyes, and around the mouth, marionette lines, and particularly deep wrinkles or creases; improving the appearance of suborbital lines and/or periorbital lines; reducing the appearance of crow's feet; rejuvenating and/or revitalizing skin, particularly aging skin; reducing skin fragility; preventing and/or reversing of loss of glycosaminoglycans and/or collagen; ameliorating the effects of estrogen imbalance; preventing skin atrophy; preventing, reducing, and/or treating hyperpigmentation or hypopigmentation; minimizing skin discoloration; improving skin tone, radiance, clarity and/or tautness; preventing, reducing, and/or ameliorating skin sagging; improving skin firmness, plumpness, suppleness and/or softness; improving procollagen and/or collagen production; improving skin texture and/or promoting retexturization; improving skin barrier repair and/or function; improving the appearance of skin contours; restoring skin luster and/or brightness; minimizing dermatological signs of fatigue and/or stress; resisting environmental stress; replenishing ingredients in the skin decreased by aging and/or menopause; improving communication among skin cells; increasing cell proliferation and/or multiplication; increasing skin cell metabolism decreased by aging and/or menopause; retarding cellular aging; improving skin moisturization; enhancing skin thickness; slowing or halting skin thinning; increasing skin elasticity and/or resiliency; enhancing exfoliation; improving microcirculation; decreasing and/or preventing cellulite formation; and any combinations thereof.

In one embodiment, the PLOD-2 stimulators will be used to reduce the severity of wrinkles, often in combination with retinol. In another embodiment, the PLOD-2 stimulators may be combined with an alpha-hydroxy (e.g., glycolic) or beta-hydroxy (e.g., salicylic) acid. In another embodiment, the composition comprises a PLOD-2 stimulator (e.g., about 0.001% to about 25% w/w), a retinoid (e.g., retinol) (e.g., about 0.01 to about 5% w/w), and glycolic acid) (e.g., about 0.001 to about 5% w/w). In yet another embodiment, the composition comprises a PLOD-2 stimulator (e.g., about 0.001% to about 25% w/w), a retinoid (e.g., retinol), and salicylic acid (e.g., about 0.01 to about 10% w/w).

In one embodiment, the compositions will comprise from about 0.00001% to about 90%, more typically from about 0.001% to about 25%, including from about 0.01 to about 10% by weight of a stimulator of PLOD-2. In one embodiment, the stimulator may be an upregulator of PLOD-2 in fibroblasts. In another embodiment, the stimulator may be an upregulator of PLOD-2 in keratinocytes. In one embodiment, the stimulator is an upregulator of PLOD-2 in both fibroblasts and keratinocytes. Combinations of stimulators are also contemplated. In one embodiment, the stimulator is a combination of two or more substances that are upregulators of PLOD-2 in fibroblasts and/or keratinocytes.

The composition will typically be applied to the skin one, two, or three times daily for as long as is necessary to achieve desired results. The treatment regiment may comprise daily application for at least one week, at least two weeks, at least four weeks, at least eight weeks, or at least twelve weeks or more. Chronic treatment regimens are also contemplated. The effect of a composition on the formation or appearance of fine lines and wrinkles can be evaluated qualitatively, e.g., by visual inspection, or quantitatively, e.g., by microscopic or computer assisted measurements of wrinkle morphology (e.g., the number, depth, length, area, volume and/or width of wrinkles per unit area of skin). In one embodiment, the composition of the invention will be applied to the skin in an amount from about 0.001 to about 100 mg/cm², typically from about 0.01 to about 20 mg/cm², and more typically about 0.1 to about 10 mg/cm².

It is also contemplated that the compositions of the invention will be useful for treating thin skin by topically applying the composition to thin skin of an individual in need thereof. “Thin skin” is intended to include skin that is thinned due to chronological aging, menopause, or photo-damage and skin that is thinning prematurely. In some embodiments, the treatment is for thin skin in men, whereas other embodiments treat thin skin in women, pre-menopausal or post-menopausal, as it is believed that skin thins differently with age in men and women, and in particular in women at different stages of life.

The method of the invention may be employed prophylactically to forestall aging including in individuals that have not manifested signs of skin aging, most commonly in individuals under 25 years of age. The method may also reverse or treat signs of aging once manifested as is common in individuals over 25 years of age, or to slow the progression of dermatological aging in such individuals.

In one embodiment, the compositions of the invention are applied to human skin to reduce sebum production or improve the appearance of skin affected by cellulite, and/or reduce unwanted lipogenesis or increase lipolysis. In this embodiment, the compounds or agents (PLOD-2 stimulators) can be formulated in cosmetically acceptable vehicles (as described herein) and may include one or more additional agents such as anti-acne ingredients (e.g., salicylic acid, benzoyl peroxide and other peroxides, sulfur, retinoids, etc.) in the case of a facial composition, or, in the case of a cellulite treatment, the formulation may comprise any ingredients suitable for treatment of cellulite, including without limitation, perilla oil and other unsaturated fatty oils and omega-3 fatty acids such as alpha-linolenic acid; caffeine; theophylline; xanthines; retinoids (e.g., retinol); and the like. A cellulite treatment according to the invention will typically be applied topically to skin suffering from cellulite, including skin of the buttocks and thighs for a period of time sufficient to improve the appearance thereof, including for example, daily treatment for at least four weeks, at least eight weeks, at least twelve weeks, or longer.

In another embodiment, the compositions of the invention are applied to human skin for depigmentation, including reducing areas of unwanted pigmentation, such as hyperpigmentation, including age spots and freckles. In this embodiment, the compounds or agents (PLOD-2 stimulators) can be formulated in cosmetically acceptable vehicles (as described herein) and may include one or more additional agents that combat pigmentation or hyperpigmentation, including tyrosinase inhibitors and/or melanosome transfer inhibitors. Special mention may be made of thiodipropionic acid and esters thereof (notably, di-lauryl esters); hydroquinone and the monobenzyl ether thereof; hydroquinone-beta-D-glucopyranoside; retinoids (e.g., retinoic acid); tretinoin; azelaic acid; Kojic acid (5-hydroxy-4-pyran-4-one-2-methyl); Mequinol (4-hydroxyanisole); Niacinamide; soy protein and other serine protease inhibitors; paper mulberry extract; Glabridin (licorice extract); Arctostaphylos patula and Arctostaphylos viscida extracts; Magnesium-L-ascorbyl-2-phosphate (MAP); 4-Isopropylcatechol; Aleosin; N-acetyl-4-S-cysteaminylphenol and N-propionyl-4-S-cysteaminylphenol; N-acetyl glucosamine; and Tranexamic acid (trans-4-aminomethylcyclohexanecarboxylic acid); to name a few.

In some embodiments, the compounds or agents of the invention inhibit PLOD-2, by which is meant that they act, through any mechanism, to diminish the cellular levels or activity of PLOD-2. The inhibitors can down-regulate expression of PLOD-2, act as antagonists of PLOD-2 or the like. PLOD-2 inhibitors are contemplated to be useful in treating conditions characterized by over production of collagen, including without limitation scleroderma. In one embodiment, the PLOD-2 inhibitors are administered topically, subcutaneously, intravenously, or orally to an individual suffering from an over production of collagen or unwanted production of collagen.

In one embodiment, the inhibitor of PLOD-2 may be a nucleic acid oligomer, typically between about 10 and about 30 nucleotides in length, more typically, between about 15 and about 25 nucleotides in length, and ideally, between 17 and 21 nucleotides in length. In one embodiment, the inhibitor of PLOD-2 is an siRNA oligomer comprising the sequence ACAUCAUGAUAGCCGUAUA (SEQ. ID 1); AAAUCUAAGUCAAGCGGAA (SEQ. ID 2); ACACAACCGAGGAGCGUAU (SEQ. ID 3); or CGGAGAAGCCCUCGAGCAU (SEQ. ID 4), or an siRNA oligomer comprising a sequence having at least 80% homology, at least 85% homology, or at least 90% homology to the foregoing. In another embodiment, the PLOD-2 inhibitor is a polyclonal or monoclonal antibody with specificity to the PLOD-2 enzyme.

In another embodiment, the compounds or agents (PLOD-2 modulators) are intended for oral use, including for pharmaceutical use. Pharmaceutical formulations will include pharmaceutically acceptable carriers (i.e., diluents and excipients). The pharmaceutical compositions may be included in solid dosage forms, including compressed tablets and capsules, or in liquid or powder forms. Pharmaceutical dosage forms will typically include from about 0.5 mg to about 200 mg, or from about 1 mg to about 100 mg of the PLOD-2 modulator. The dosage forms may be immediate release, in which case they will typically comprise a water-soluble or dispersible carrier such as microcrystalline cellulose, mannitol, hydroxypropyl methyl cellulose, PVP or the like, or may be delayed, sustained, or modified release, in which case they may comprise water-insoluble polymers such as cellulose ethers (e.g., ethylcellulose), alone or in combination with water soluble or dispersible polymers, to regulate the rate of dissolution of the dosage form in the stomach.

### EXAMPLES

The following examples describe specific aspects of the invention to illustrate the invention but should not be construed as limiting the invention, as the examples merely provide specific methodology useful in the understanding and practice of the invention and its various aspects.

### Example 1

### PLOD-2 Expression Declines with Age

Cell Treatment:

Normal Human dermal fibroblasts (donor cells from three young donors, average age 20 years, and from three older donors, average age 60 years) were grown in DMEM (Mediatech; cat. #: 15-013-CV) containing 10% Fetal Bovine Serum (Perbio; cat. #: SH30070.03), Penicillin/Streptomycin (Mediatech, Cat #30-001-C1), L-Glutamine (Mediatech; cat. #: 25-005-CI) at 37° C. and 10% CO₂. Cells were grown to about 80% confluence at P4/P5. RNA was isolated using RNeasy RNA extraction kit (74106) from Qiagen. RNA concentrations were determined using NanoDrop Spectrophotometer ND 1000 (Agilent Technologies).

Reverse Transcription (RT):

Equal concentrations of RNA from three young donors (average age 20 years) and from three older donors (average age 60 years) were pooled for analysis. RT reactions were conducted in a total volume of 20 n1 using High Capacity cDNA kit from AB (PN 4368814). The RT mixture was prepared to contain 2 μl 10× TaqMan RT buffer, 1.2 n1 dNTP mix (100 nm), 1.0 μl 10× Random Hexamer, 1 μl RNase Inhibitor, 1 μl MultiScribe Reverse Transcriptase (50 U/ml), 100 ng of RNA, and RNase-free water to make up the final volume of 20 μl. The reaction was incubated at 25° C. for 10 min, 45° C. for 45 min, and then 95° C. for 5 min in a BIORAD MY CYCLER.

Polymerase Chain Reaction (PCR):

QPCR was carried out using Applied Biosystems Universal PCR Master Mix (PN 4369016). The mixture contained 10 n1 of Taqman Universal PCR mix, 1 μl of primer and probe mix, 2 μl of RT product, and 7 μl of deionized water. All probes, Taqman assays were, were purchased from Applied Biosystems, Hs01118190_m1 for PLOD-2, and human GAPDH (PN 4352934). The temperature profiles for qPCR were 50° C. for 2 min, and 95° C. for 10 min for 1 cycle, then at 95° C. for 15 sec, and 60° C. for 1 min for 40 cycles carried out in Stratagene Mx 3005P qPCR machine.

Results:

The age related expression of PLOD-2 in younger versus older skin fibroblasts is examined. Data in Table 1 demonstrates that there is a significantly lower level of PLOD-2 in older compared to younger skin fibroblasts. All values are statistically significant at p<0.05.

TABLE 1

| Age | Relative PLOD-2 Level |
|---|---|
| Young | 100% |
| Old | 27% |

PLOD-2 gene expression significantly declines with age in normal human dermal fibroblasts, in vitro. Since PLOD-2 modification is critical for the stability of procollagen, a decline in PLOD-2 expression can lead to a decrease in collagen production; thereby resulting in less support in the dermal matrix and therefore contribute to wrinkle formation.

### Example 2

### PLOD-2 Levels Modulate Collagen Production

Cell Treatment:

Young normal Human dermal fibroblasts (˜22 yrs) were grown in DMEM (Mediatech; cat. #: 15-013-CV) containing 10% Fetal Bovine Serum (Perbio; cat. #: SH30070.03), Penicillin/Streptomycin (Mediatech, Cat #30-001-C1), L-Glutamine (Mediatech; cat. #: 25-005-CI) at 37° C. and 10% CO₂. Small interference RNA (siRNA) On-TARGETplus siRNA against PLOD-2 (Sequences: ACAUCAUGAUAGCCGUAUA; AAAUCUAAGUCAAGCGGAA; ACACAACCGAGGAGCGUAU CGGAGAAGCCCUCGAGCAU) (Dharmacon Inc, Human, L-004285-01-0005) or siCONTROL nontargeting/scrambled siRNA (Dharmacon D001810-10-05) were transfected into cells at a final concentration of 50 nM using Lipofectamine (Invitrogen, 12252-011) following manufacturer's protocol. 72 hrs post-transfection RNA was isolated using RNeasy RNA extraction kit (74106) from Qiagen. RNA concentrations were determined using NanoDrop Spectrophotometer ND 1000 (Agilent Technologies). Conditioned Tissue culture medium was collected for procollagen analysis.

Reverse Transcription (RT):

RT reactions were conducted in a total volume of 20 μl using High Capacity cDNA kit from AB (PN 4368814). The RT mixture was prepared to contain 2 μl 10× TaqMan RT buffer, 1.2 μl dNTP mix (100 nm), 1.0 μl 10× Random Hexamer, 1 μl RNase Inhibitor, 1 μl MultiScribe Reverse Transcriptase (50 U/ml), 100 ng of RNA, and RNase-free water to make up the final volume of 20 μl. The reaction was incubated at 25° C. for 10 min, 45° C. for 45 min, and then 95° C. for 5 min in a BIORAD MY CYCLER.

Polymerase Chain Reaction (PCR):

QPCR was carried out using Applied Biosystems Universal PCR Master Mix (PN 4369016). The mixture contained 10 μl of Taqman Universal PCR mix, 1 μl of primer and probe mix, 2 μl of RT product, and 7 μl of deionized water. All probes, Taqman assays were, were purchased from Applied Biosystems, Hs01118190 ml for PLOD-2, and human GAPDH (PN 4352934). The temperature profiles for qPCR were 50° C. for 2 min, and 95° C. for 10 min for 1 cycle, then at 95° C. for 15 sec, and 60° C. for 1 min for 40 cycles carried out in Stratagene Mx 3005P qPCR machine.

Procollagen Assay:

Procollagen levels in the conditioned medium were determined using ProcollagenType-1 C-Peptide EIA Kit from Takara; cat. # MK101 as per manufacturer's suggestions. Reading was measured at 450 nm using a spectrophotometer.

Results:

Human dermal fibroblasts (age—22 yrs) were treated with siRNA against PLOD-2 for 72 hrs showed a signification decrease in PLOD-2 mRNA compared to cells treated with control siRNA. In addition, the conditioned medium from samples treated with siRNA against PLOD-2 demonstrated a significant decrease in procollagen levels, relative to control treated cells, as shown in Table 2. These data indicate that suppression of PLOD-2 leads to a decrease in the level of procollagen production. All values are statistically significant at p<0.05.

TABLE 2

| | Relative PLOD-2 | Relative Procollagen |
|---|---|---|
| Treatment | Expression | Level |
| Control | 100% | 100% |
| (Scrambled siRNA 50 nM) | | |
| PLOD-2 siRNA 50 nM | 20% | 85% |

### Example 3

### PLOD-2 Enzyme Stimulation Assay

Cell Treatment:

Normal Human dermal fibroblasts were grown in DMEM (Mediatech; cat. #: 15-013-CV) containing 10% Fetal Bovine Serum (Perbio; cat. #: SH30070.03), Penicillin/Streptomycin (Mediatech, Cat #30-001-C1), L-Glutamine (Mediatech; cat. #: 25-005-CI) at 37° C. and 10% CO₂. Cells were stripped of serum overnight, followed by treatment with 0.05% N-Acetyl-Tyrosinamide, ALB-H09784893, ALB-H16063163, or vehicle (DMSO), in the absence of serum for 48 hours. RNA was isolated using RNeasy RNA extraction kit (74106) from Qiagen. RNA concentrations were determined using NanoDrop Spectrophotometer ND 1000 (Agilent Technologies).

Reverse Transcription (RT):

RT reactions were conducted in a total volume of 20 μl using the High Capacity cDNA kit from AB (PN 4368814). The RT mixture was prepared to contain 2 μl 10× TaqMan RT buffer, 1.2 μl dNTP mix (100 nm), 1.0 μl 10× Random Hexamer, 1 μl RNase Inhibitor, 1 μl MultiScribe Reverse Transcriptase (50 U/ml), 100 ng of RNA, and RNase-free water to make up the final volume of 20 μl. The reaction was incubated at 25° C. for 10 min, 45° C. for 45 min, and then 95° C. for 5 min in a BIORAD MY CYCLER.

Polymerase Chain Reaction (PCR):

QPCR was carried out using Applied Biosystems Universal PCR Master Mix (PN 4369016). The mixture contained 10 μl of Taqman Universal PCR mix, 1 μl of primer and probe mix, 2 μl of RT product, and 7 μl of deionized water. All probes, Taqman assays were, were purchased from Applied Biosystems, Hs01118190 m1 for PLOD-2, and human GAPDH (PN 4352934). The temperature profiles for qPCR were 50° C. for 2 min, and 95° C. for 10 min for 1 cycle, then at 95° C. for 15 sec, and 60° C. for 1 min for 40 cycles carried out in Stratagene Mx 3005P qPCR machine.

Results:

Cells treated with a solution of 0.05% N-Acetyl-Tyrosinamide, 0.0005% ALB-H09784893, and 0.0005% ALB-H16063163, respectively, demonstrated a significant increase in PLOD-2 levels, relative to control, vehicle treated cells, as shown by the data in Table 3. Data represents an average of two or three independent experiments. All values are statistically significant at p<0.05.

TABLE 3

| Treatment | Relative PLOD-2 Expression |
|---|---|
| Vehicle (DMSO) | 100% |
| N-Acetyl-Tyrosinamide (0.05%) | 180% |
| ALB-H09784893 (0.0005%) | 125% |
| ALB-H16063163 (0.0005%) | 125% |

These results show that N-Acetyl-Tyrosinamide, ALB-H09784893 and ALB-H16063163 stimulate the expression of PLOD-2 in normal Human Dermal Fibroblasts, in vitro. Because PLOD-2 modification is critical for the stability of procollagen, a key building blocks of the dermis, a decline in PLOD-2 expression can lead to a decrease in collagen production, thereby resulting in the weakening of the dermal matrix and therefore contribute to wrinkle formation. The data in Table 3 shows that N-Acetyl-Tyrosinamide, ALB-H09784893 and ALB-H16063163 can help to intercept critical collagen blockers to increase skin's key building blocks and thus generate new collagen to help fill wrinkles

### Example 4

### Stimulation of Collagen, Fibrillin and Elastin Production

Cell Treatment:

Normal Human dermal fibroblasts were grown in DMEM (Mediatech; cat. #: 15-013-CV) containing 10% Fetal Bovine Serum (Perbio; cat. #: SH30070.03), Penicillin/Streptomycin (Mediatech, Cat #30-001-C1), L-Glutamine (Mediatech; cat. #: 25-005-CI) at 37° C. and 10% CO₂. Cells were stripped of serum overnight, followed by treatment with a solution containing 0.05% N-Acetyl-Tyrosinamide, 0.0005% ALB-H09784893, 0.0005% ALB-H16063163, or vehicle (DMSO), in the absence of serum for 48 hours. RNA was isolated using RNeasy RNA extraction kit (74106) from Qiagen. RNA concentrations were determined using NanoDrop Spectrophotometer ND 1000 (Agilent Technologies).

Reverse Transcription (RT):

RT reactions were conducted in a total volume of 20 μl using High Capacity cDNA kit from AB (PN 4368814). The RT mixture was prepared to contain 2 μl 10× TaqMan RT buffer, 1.2 μl dNTP mix (100 nm), 1.0 μl 10× Random Hexamer, 1 μl RNase Inhibitor, 1 μl MultiScribe Reverse Transcriptase (50 U/ml), 100 ng of RNA, and RNase-free water to make up the final volume of 20 μl. The reaction was incubated at 25° C. for 10 min, 45° C. for 45 min, and then 95° C. for 5 min in a BIORAD MY CYCLER.

Polymerase Chain Reaction (PCR):

QPCR was carried out using Applied Biosystems Universal PCR Master Mix (PN 4369016). The mixture contained 10 μl of Taqman Universal PCR mix, 1 μl of primer and probe mix, 2 μl of RT product, and 7 μl of deionized water. All probes, Taqman assays were, were purchased from Applied Biosystems, COL1a—Hs00164004_m1, ELN—Hs00355783_m1, FBN1—Hs00171191_m1, and human GAPDH (PN 4352934). The temperature profiles for qPCR were 50° C. for 2 min, and 95° C. for 10 min for 1 cycle, then at 95° C. for 15 sec, and 60° C. for 1 min for 40 cycles carried out in Stratagene Mx 3005P qPCR machine.

Procollagen Assay:

Procollagen levels in the conditioned medium were determined using ProcollagenType-1 C-Peptide EIA Kit from Takara; cat. # MK101 as per manufacturer's suggestions. Reading was measured at 450 nm using a spectrophotometer.

Results:

Human dermal fibroblasts treated with 0.05% N-Acetyl-Tyrosinamide (Table 4a), for 48 hours were analyzed by qRT-PCR for expression levels of COL 1a, ELN and FBN1, using GAPDH as an internal control, and demonstrated a significant increase in COL1a, relative to control, vehicle treated cells. Cells treated with N-Acetyl-Tyrosinamide also showed a significant increase in ELN and FBN1 expression as shown in Table 4a.

Human dermal fibroblasts treated with 0.0005% ALB-H09784893 and 0.0005% ALB-H16063163 for 48 hours were analyzed by ELISA for expression levels of pro-collagen. Human dermal fibroblasts treated with 0.0005% ALB-H09784893 and 0.0005% ALB-H16063163 demonstrated a significant increase in pro-collagen, relative to control, vehicle treated cells (Table 4b).

All values are statistically significant at p<0.05.

TABLE 4a

| | Pro- | | |
|---|---|---|---|
| | collagen | Elastin | Fibrillin |
| N-Acetyl-Tyrosinamide (0.05%) | 16% | 29% | 29% |

TABLE 4b

| | Pro- |
|---|---|
| | collagen |
| ALB-H09784893 (0.0005%) | 46% |
| ALB-H16063163 (0.0005) | 46% |

Collagen, Elastin and Fibrillin are key building blocks of the dermis, decline with age, and thus contribute to wrinkle formation. The data in Table 4a-b shows that N-Acetyl-Tyrosinamide Acetyl, ALB-H09784893, and ALB-H16063163 help increase pro-collagen, which will generate new collagen to help fill wrinkles

### Example 5

### N-Acetyl-Tyrosinamide, ALB-H09784893, and ALB-H16063163 Versus Other Anti-Aging Actives

Cell Treatment:

Normal Human Dermal Fibroblasts were Grown in DMEM (Mediatech; cat. #: 15-013-CV) containing 10% Fetal Bovine Serum (Perbio; cat. #: SH30070.03), Penicillin/Streptomycin (Mediatech, Cat #30-001-C1), L-Glutamine (Mediatech; cat. #: 25-005-CI) at 37° C. and 10% CO₂. Cells were stripped of serum overnight, followed by treatment with Test Actives at indicated concentration (maximum dose that does not induce cell toxicity) as listed in Table 5, in the absence of serum for 48 hours. RNA was isolated using RNeasy RNA extraction kit (74106) from Qiagen. RNA concentrations were determined using NanoDrop Spectrophotometer ND 1000 (Agilent Technologies).

Reverse Transcription (RT):

RT reactions were conducted in a total volume of 20 μl using the High Capacity cDNA kit from AB (PN 4368814). The RT mixture was prepared to contain 2 μl 10× TaqMan RT buffer, 1.2 μl dNTP mix (100 nm), 1.0 μl 10× Random Hexamer, 1 μl RNase Inhibitor, 1 μl MultiScribe Reverse Transcriptase (50 U/ml), 100 ng of RNA, and RNase-free water to make up the final volume of 20 μl. The reaction was incubated at 25° C. for 10 min, 45° C. for 45 min, and then 95° C. for 5 min in a BIORAD MY CYCLER.

Polymerase Chain Reaction (PCR):

QPCR was carried out using Applied Biosystems Universal PCR Master Mix (PN 4369016). The mixture contained 10 μl of Taqman Universal PCR mix, 1 μl of primer and probe mix, 2 μl of RT product, and 7 μl of deionized water. All probes, Taqman assays were, were purchased from Applied Biosystems, Hs01118190 ml for PLOD-2, and human GAPDH (PN 4352934). The temperature profiles for qPCR were 50° C. for 2 min, and 95° C. for 10 min for 1 cycle, then at 95° C. for 15 sec, and 60° C. for 1 min for 40 cycles carried out in Stratagene Mx 3005P qPCR machine.

Results:

Human dermal fibroblasts (age—57 yrs) were grown in the absence of serum overnight, followed by treatment as indicated in Table 5, in the absence of serum for 48 hours. RNA isolated from cells were analyzed by qRT-PCR for expression levels of PLOD-2, using GAPDH as an internal control. Cells treated with leading anti-aging ingredients do not stimulate PLOD-2 levels, relative to control, vehicle treated cells, as shown in Table 5. However, cells treated with a composition comprising 0.05% N-Acetyl-Tyrosinamide, 0.0005% ALB-H09784893, and 0.0005% ALB-H16063163, robustly induce PLOD-2 genes expression, p<0.05.

TABLE 5

| | | Significant Increases in |
|---|---|---|
| Test Active | Concentration | PLOD-2 Expression |
| N-Acetyl-Tyrosinamide | 0.05% | Yes (80%) |
| ALB-H09784893 | 0.0005% | Yes (25%) |
| ALB-H16063163 | 0.0005% | Yes (25%) |
| Injectable Collagen | 0.05% | No |
| | 0.005% | No |
| Glycolic Acid (AHA) | 0.10% | No |
| | 0.05% | No |
| Matrixyl | 0.10% | No |
| | 0.05% | No |
| Niacinamide | 0.10% | No |
| | 0.05% | No |
| Hyaluronic Acid | 0.005% | No |
| | 0.001% | No |
| Resveratrol | 5 μM | No |
| | 10 μM | No |
| Retinol | 5 μM | No |
| | 10 μM | No |
| Coenzyme Q10 | 5 μM | Yes (11%) |
| | 10 μM | Yes (24%) |

The results in Table 5 show that several actives commonly used in anti-aging products including Injectable Collagen, Glycolic acid, Matrixyl, Niacinamide, Hyaluronic acid, Reseveratrol, and Retinol did not induce a significant level of PLOD-2 in skin cells, while N-Acetyl-Tyrosinamide, ALB-H09784893, and ALB-H16063163 significantly stimulate the level of PLOD-2. This study shows that N-Acetyl-Tyrosinamide, ALB-H09784893 and ALB-H16063163 act in a manner that is different from that of other popular anti-aging actives such Retinol, AHA and injectable collagen.

All references including patent applications and publications cited herein are incorporated herein by reference in their entirety and for all purposes to the same extent as if each individual publication or patent or patent application was specifically and individually indicated to be incorporated by reference in its entirety for all purposes. Many modifications and variations of this invention can be made without departing from its spirit and scope, as will be apparent to those skilled in the art. The specific embodiments described herein are offered by way of example only, and the invention is to be limited only by the terms of the appended claims, along with the full scope of equivalents to which such claims are entitled.

1. A cosmetic composition for improving the aesthetic appearance of human skin comprising a cosmetically acceptable vehicle, and an effective amount of an active agent that upregulates PLOD-2 by at least 25%, with the proviso that said active agent is not N-Acetyl-Tyrosinamide.

2. The cosmetic composition according to claim 1, wherein the vehicle is in the form of a water-in-oil, oil-in-water, silicone-in-water, water-in-silicone, polyol-in-silicone, or silicone-in-polyol emulsion.

3. The cosmetic composition according to claim 1, further comprising a retinoid.

4. A method for improving the aesthetic appearance of human skin comprising topically applying to an area of the skin in need thereof an effective amount of an active agent, other than N-Acetyl-Tyrosinamide, that stimulates PLOD-2 expression, wherein said active agent upregulates PLOD-2 by at least 25%, for a time sufficient to improve the aesthetic appearance of said human skin.

5. The method according to claim 4, wherein said aesthetic improvement of said human skin is selected from the group consisting of:
(a) treatment, reduction, and/or prevention of fine lines or wrinkles;
(b) reduction of skin pore size;
(c) improvement in skin thickness, plumpness, and/or tautness;
(d) improvement in skin smoothness, suppleness and/or softness;
(e) improvement in skin tone, radiance, and/or clarity;
(f) improvement in procollagen, and/or collagen production;
(g) improvement in maintenance and remodeling of elastin;
(h) improvement in skin texture and/or promotion of retexturization;
(i) improvement in skin barrier repair and/or function;
(j) improvement in appearance of skin contours;
(k) restoration of skin luster and/or brightness;
(l) replenishment of essential nutrients and/or constituents in the skin;
(m) improvement of skin appearance decreased by aging and/or menopause;
(n) improvement in skin moisturization;
(o) increase in skin elasticity and/or resiliency;
(p) treatment, reduction, and/or prevention of skin sagging;
(q) improvement in skin firmness; and
(r) reduction of pigment spots and/or mottled skin; and
(s) improvement of optical properties of skin by light diffraction or reflection.

6. A method for improving the aesthetic appearance of human skin comprising topically applying to an area of the skin in need thereof a composition comprising a retinoid and an effective amount of at least one active agent that modulates cellular levels of PLOD-2 other than N-Acetyl-Tyrosinamide.

7. The method according to claim 6, wherein the ability to modulate cellular levels of PLOD-2 is determined by measuring the expression level of mRNA encoding PLOD-2 in a dermal fibroblast or keratinocyte.

8. A method for screening active agents useful for improving the aesthetic appearance of skin comprising assaying candidate substances for ability to stimulate PLOD-2 expression in a dermal fibroblast or keratinocyte.

9. The method according to claim 8, wherein said assaying step comprises incubating human dermal fibroblasts or keratinocytes with said candidate substance and subsequently measuring the levels of mRNA encoding PLOD-2.

10. The method according to claim 9, wherein said step of measuring is carried out by quantitative polymerase chain reaction (qPCR).

11. A method of treating the skin comprising topically applying to an area of the skin in need thereof an effective amount of an active agent that modulates PLOD-2 expression, other than N-Acetyl-Tyrosinamide, wherein the ability of said active agent to modulate PLOD-2 has been determined by an assay which measures PLOD-2 expression in a dermal fibroblast or keratinocyte.

12. The method according to claim 11, wherein said assaying step comprises incubating human dermal fibroblasts with said candidate substance and subsequently measuring the levels of mRNA encoding PLOD-2.

13. The method according to claim 12, wherein the ability to modulate PLOD-2 are determined by measuring the expression levels of the corresponding mRNA levels of PLOD-2.

14. The method according to claim 11, wherein said agent increases expression levels of PLOD-2.

15. The method according to claim 11, wherein said agent decreases expression levels of PLOD-2.

16. The method according to claim 11, wherein said method comprises the aesthetic improvement of said skin and the treatment, reduction, and/or prevention of fine lines and/or wrinkles, skin sagging, and loss of elasticity.

17. The method according to claim 16, wherein said aesthetic improvement of said skin is the treatment of wrinkles and/or fine lines on the skin, and wherein said method comprises topically applying an effective amount of an active agent in a cosmetically acceptable vehicle to affected skin of an individual in need thereof, for a time sufficient to reduce the severity of said wrinkles and/or fine lines.

18. The method according to claim 16, wherein said active agent is applied to said skin at least once daily for a period of at least four weeks.

19. A method for improving the aesthetic appearance of human skin comprising topically applying to an area of the skin in need thereof an effective amount of retinol in combination with an active agent that modulates cellular levels of PLOD-2, other than N-Acetyl-Tyrosinamide, wherein the ability of said active agent to modulate PLOD-2 has been determined by an assay which measures the expression level of PLOD-2 in a cell that has been contacted with said active agent.
